(19) United States
(12) Patent Application Publication
(10) Pub. No.: US 2007/0071703 A1
(43) Pub. Date: Mar. 29, 2007

(54) High efficiency master gels for thickening oil based compositions

(76) Inventor: Samuel Qcheng Sun Lin, Paramus, NJ (US)

Correspondence Address: Michael P. Aronson;MP&G Aronson Inc., 2 Mandarin Lane, West Nyack, NY, 10994, US

(21) Appl. No.: 11/237,293

(22) Filed: Sep. 28, 2005

(51) Int. Cl. A61K 8/97; A61K 8/89

(52) U.S. Cl. 424/70.12; 424/74

(57) ABSTRACT

The composition, preparation and use of a thickening system or master gel for oil based compositions, especially cosmetic compositions is described. These highly efficient master gels include: i) greater than about 10% to about 40% of an organoclay, ii) from about 70% to less than about 90% of a carrier oil comprising a substantially dissolved mixture of at least one silicone fluid and at least one organic oil wherein the total silicone fluids comprise from about 10% to about 90% of the carrier based on the total weight of the carrier, and ii) from 0.5% to about 6% of a polar activator. In a preferred embodiment the master gel contains from about 15% to about 30% of an organoclay and is chosen so that the viscosity of the master gel composition is at least 2.5 million cP as measured with a Brookfield viscometer at 1 rpm.

### FIELD OF INVENTION

The invention relates to the preparation and use of thickening systems or master gels that are used to thicken or structure oil based compositions. The inventive master gels are highly efficient and are especially useful in cosmetic applications.

### BACKGROUND OF INVENTION

Organoclays are produced by reacting organic cations such as di-(C₁₆-C₂₀ alkyl) ammonium chloride with clays through an ion-exchange mechanism. These organoclays are known in the art to swell or exfoliate in certain organic liquids, resulting in gelled organic liquids; see for example, “Clay Mineralogy”, 2nd edition, 1968, by Ralph Grim, published by McGraw Hill Book Co. These gels are useful as lubricating greases, oil based muds, paints, foundry molding sand binders, etc.

Recently, their applications have been extended to personal care and cosmetic products. However, in order to produce gels that are useful, consistent, and acceptable in the personal care and cosmetic industry, the organoclay powder has to be dispersed in cosmetic oils, and activated or exfoliated fully using intensive shear mixing equipments and polar activators over a long mixing time. This energy intensive and time-consuming process is inconvenient and ineffective in the manufacturing of high-valued personal care and cosmetic products. To overcome this problem, thickening concentrates or “master gels” have been utilized. The term “master gel” is used herein to designate a pre-activated and concentrated dispersion of an organoclay in cosmetically acceptable oil.

As an intermediate cosmetic raw material, master gels can be incorporated easily into the formulation during the manufacture of a personal care and cosmetic product using low to medium shear mixing, thereby overcoming the shortfall of organoclay powder, i.e., eliminating the intensive mixing steps and associated problems in handling powders and viscous phases.

There are a number of commercial master gels utilizing organoclays in combination with propylene carbonate as a polar activator in various cosmetic oils. For example, Elementis Specialties at Hightstown, N.J. 08520, USA markets BENTONE GEL VS5V and VS5-PCV (disteardimonium hectorite in cyclopentasiloxane), BETONE GEL IHDV (disteardimonium hectorite in isohexadecane), BENTONE GEL GTCC V (Stearalkonium hectorite in caprylic/capric triglyceride), and BENTONE GEL TNV (Stearalkonium in C₁₂₋₁₅ alkyl benzoate). Sud-Chemie Inc., at 1600 West Hill Street, Louisville, Ky. 40210, markets TIXOGEL VSP (Stearalkonium-90 bentonite in cyclopentasiloxane), TIXOGEL IHD (stearalkonium-90 bentonite in isohexadecane), and TIXOGEL FTN (stearalkonium-90 bentonite in C₁₂₋₁₅ alkyl benzoate).

The commercially available gels are generally designed for thickening the specific types of cosmetic oils that are used as their carriers, and have varying efficacy when used for oils outside their intended oils. For example, Bentone Gel VS5V and Tixogel VSP containing only volatile cyclopentasiloxane as their carrier and are designed by those skilled in the art for thickening the volatile cyclopentasiloxane and other similar volatile silicone oils. Tixogel FTN containing C₁₂₋₁₅ alkyl benzoate organic ester oil as its carrier is designed for thickening the C₁₂₋₁₅ alkyl benzoate oil.

However, the formulations of the modern commercial cosmetic and toiletry products are comprised of up to 50 components, including mixtures of oils. In practice, formulation chemists are forced to investigate all commercial master gels in their developmental formulations, which is a time consuming process and is often impractical. Thus, there is a need for a universal master gel that is capable of thickening a wide range of silicone oils, organic oils and their mixtures.

Furthermore, the commercial master gels for many oils are not very efficient and a high % level in the formulation is often required to deliver adequate thickening. It is particularly difficult to thicken low molecular weight silicone oils, which have highly preferred properties such as providing a silky and lubricous sensory feel to the composition; the commercial master gels such as BENTONE GEL VS5V and TIXOGEL VSP are designed for volatile silicones, they are still not very effective nor efficient and high levels are required.

The formulations of the modern cosmetic and toiletry products are complex and can be comprised of numerous components (>40) for delivering a range of consumer benefits such as moisturization, anti-itching, anti-aging, broad spectrum UV protection, color, to name a few. Thus, there is a need for a master gel that is highly efficient (high thickening at low inclusion in of the formulation), thereby providing more formulation space for critical actives or components.

Thus, there has been an unmet need for master gel compositions that are highly efficient at low % level and universally effective in a variety of oil based systems including both organic oils, silicones, and their mixtures.

The following patents and publications form a part of the related art:

U.S. Pat. No. 4,526,780 (also GB 2096891) to Marschner et al Issued Jul. 2, 1985 discloses anhydrous antiperspirant compositions in paste and cream form including an oil absorbing material and a clay suspending/thickening agent in a volatile silicone.

U.S. Pat. No. 4,425,328 to Nabial issued Jan. 10, 1984 discloses a solid antiperspirant stick compositions that includes hydrophobic waxy materials as structuring agents and volatile silicone in combination with polyoxypropylene-alkyl esters as emollients. Organoclay is disclosed as a suspending agent.

US 2004/0122152 to SenGupta et al published Jun. 24, 2004 discloses compositions for thickening hydrophobic liquids containing layered silicate materials having its surface modified by an adsorbed amphiphilic copolymer. The silicates include only the hydrophilic clays.

U.S. Pat. No. 5,939,475 to Reynolds et al issued Aug. 17, 1999 discloses a pourable compositions useful as a rheological additive for organic fluid systems that includes an organophilic clay, a polyamide, and an organic solvent.

U.S. Pat. No. 2,677,661 to O'Halloran issued May 4, 1954 discloses bentonite greases as lubricating compositions. A solvent exchange process is disclosed to prepare organophilic clay.

U.S. Pat. No. 3,294,683 to Stansfield et al issued Dec. 27, 1966 discloses “water proofed” clay-thickened grease compositions which are capable of use in high-temperature applications.

The present invention seeks improvements over deficiencies in the known art. Among the one or more problems addressed include the development of a master gel composition that is highly efficient and versatile for structuring a range of cosmetic oils.

### SUMMARY OF THE INVENTION

The present invention describes an improved thickening system or master gel for thickening or structuring oil based systems especially for cosmetic applications. The inventor has found through numerous experiments with organoclay dispersions that the composition of the oil carrier has a pronounced effect on the rheological properties of the dispersion. By using a carrier that incorporates a compatible mixture of both a silicone and organic fluid, it has been found possible to process organoclay dispersions on a practical scale that have higher clay loadings and can efficiently thicken a range of oils. Their versatility and efficiency as well as their silky sensory properties make these inventive master gel compositions especially useful as cosmetic intermediates.

More specifically, these versatile master gel compositions include:
i) greater than about 10% to about 40% of an organoclay,
ii) from about 64% to about 89% of a carrier oil comprising a substantially dissolved mixture of at least one silicone fluid and at least one organic oil wherein the total silicone fluids comprise from about 10% to about 90% of the carrier based on the total weight of the carrier,
iii) from 0.5 to about 6% of a polar activator.

In a preferred embodiment the master gel contains 15% to about 35% of organo clay and is chosen so that the viscosity of the master gel composition is at least 2.5 million cP as measured with a Brookfield viscometer at 1 rpm.

Another embodiment of the invention relates to a method of thickening oil-based compositions, especially cosmetic oil-based compositions that entails incorporating the inventive master gel into the cosmetic formulation at a level of from about 1% to about 30% by weight of the cosmetic composition.

These and other variations of the inventive compositions disclosed herein will become clear from the description of the invention which follows.

### DETAILED DESCRIPTION OF THE INVENTION

As used herein % or wt % refers to percent by weight of an ingredient as compared to the total weight of the composition or component that is being discussed.

Except in the operating and comparative examples, or where otherwise explicitly indicated, all numbers in this description indicating amounts of material or conditions of reaction, physical properties of materials and/or use are to be understood as modified by the word “about.” All amounts are by weight of the final composition, unless otherwise specified.

For the avoidance of doubt the word “comprising” is intended to mean “including” but not necessarily “consisting of” or “composed of.” In other words, the listed steps or options need not be exhaustive.

The invention relates to the composition, preparation and use of oil-based thickening concentrates which are herein designated as “master gels”. These master gels are used to thicken or structure oil based compositions especially oil based compositions intended for cosmetic applications.

The master gel compositions of the invention include three key components each of which can be a mixture: organoclay, carrier oil (alternatively designated as simply “carrier”), and a polar activator (alternatively designated “activator”). These components, other optional ingredients and methods of preparation and evaluation of the inventive master gels are described in detail below.

Organoclay

Organoclays suitable for this invention are derived from reaction of smectite clays with organic compounds.

The term “smectite clay” is used to describe a family of expansible 2:1 phyllosilicate clay minerals (layered silicates) having permanent layer charge because of the isomorphous substitution in either the octahedral sheet (typically from the substitution of low charge species such as Mg²⁺, Fe²⁺, or Mn²⁺ for Al³⁺) or the tetrahedral sheet (where Al³⁺ or occasionally Fe³⁺ substitutes for Si⁴⁺). It is common for smectites to have both tetrahedral charge and octahedral charge.

These isomorphous substitutions lead to net negative charges on the clay structure which must be satisfied by the presence of charge-balancing cations somewhere else in the structure. In the natural state, the interlayer is hydrated, which allows cations to move freely in and out of the structure. Because the interlayer is open and hydrated, cations may be present within the interlayer to balance negative charges on the sheets themselves. These cations between the layers are the predominant part of the cation exchange capacity (CEC) of the clay. Smectite clays will have a CEC of around 80 to 150 meq/100 g. These cations can also be exchanged with nonpolar organic cations, which renders the sheets expandable in organic solvents.

The interlayer in smectites is not only hydrated, but it is also expansible; that is, the separation between individual smectite sheets varies depending on: i) the type of interlayer cations present (monovalent cations like Na⁺ cause more expansion than do divalent cations like Ca²⁺), ii) the concentration of ions in the surrounding solution, and iii) the amount of water present.

Because the interlayer is expansible, smectites are often referred to as “swelling clays”. Soils having high concentrations of smectites can undergo as much as a 30% volume change due to wetting and drying, i.e., these soils have a high shrink/swell potential.

Smectite clays include the minerals montmorillonite, an aluminium silicate and hectorite, a magnesium silicate. Benonite is a smectite clay primarily composed of montmorrillonite and is named after the Benton Formation (formerly Fort Benton Formation) of the Rock Creek district in eastern Wyoming. However, the terms Bentonite, smectite and montmorillonite are sometimes used interchangeably in the art to designate swelling clays that are useful in the present context as thickeners. As used herein, “Bentonite” denotes the class of swelling clays that encompasses smectite clays, montomorillonite, hectorite and the like.

Because Bentonites are composed of layered hydrated sheets they delaminate and swell readily in water but not in organic solvents of low polarity. However, if the clay is reacted with a cationic organic compound which adsorbs at the negatively charged faces of the inlayer sheets (ion exchange), the clay will delaminate and swell in and thicken organic liquids.

The organic compounds that have been found to be most suitable are typically quaternary ammonium salts containing methyl, or benzyl radical, and a mixture of dialkyl or trialkyl radicals having from 14 to 30 carbon atoms in the chain.

The organoclays can be prepared by mixing together the clay, quaternary ammonium compound, and water for a period sufficient for the ion-exchange to take place, followed by filtering, washing, and grinding. Details of the preparation and chemistry can be found in U.S. Patents: U.S. Pat. No. 2,966,506; U.S. Pat. No. 3,974,125; U.S. Pat. No. 3,537,994; U.S. Pat. No. 5,739,087; and U.S. Pat. No. 5,718,841, and in the publication “Rheology Handbook, A Practical Guide” by Elementis Specialties all incorporated by reference herein. Self-activating organoclays as described in U.S. Pat. No. 5,379,087 are a special class of organoclays from reacting smectite clays with a larger amount of organic quaternary ammonium compounds at 120-160 milli equivalents per 100 g of the clay, 100% active clay basis. The self-activating organoclays typically contain a very high % weight of organic ammonium compounds; and they require no or less amount of activators for exfoliation.

Commercially available organoclays include, for example, TIXOGEL from Sud-Chemie, BENTOGEL ^{from} Elementis, and CLAYTONE and GARAMITE from Southern Clay Products. GARAMITE LS is a self-activating organoclay. CLOSITE clay powders, from Southern Clay Products, Inc., are the organoclays containing a very high Wt % of organic ammonium compounds. CLOSITE 25 A is a natural montmorillonite modified with 34% by weight of dimethyl hydrogenated tallow ethylhexyl ammonium methyl sulfate; CLOISITE 93A contains 40% by weight of methyl dehydrogenated tallow ammonium methyl sulfate. NANOMER organoclay powders from NANOCOR also contain high Wt % of organic ammoniums.

The level of organoclay in the mater gel should be greater than about 10% up to about 40%, preferably between about 15% and about 35%, and most preferably about 15% and 30% by weight of the master gel composition.

Carrier Oil

The term carrier oil or carrier is used to designate the predominant component or components that makeup the non-aqueous solvent or oil phase in which the organoclay is dispersed. The ideal carrier for the invention should have several desirable properties. First, an optimal carrier should allow a high degree of swelling and delaminating of the hydrophobic clay platelets so that the organoclay achieves a high degree of efficiency either self-activated or when activated by the polar activator used in the composition to assist delamination (see below).

A second desirable property of the carrier is related to the rate of swelling and delamination of the organoclay during the dispersing process. If delamination is too rapid, the viscosity of the master gel rises so quickly that the dispersion can not be adequately mixed before the power requirements become too large for the mixer to handle. When this happens, part of the organoclay is not adequately dispersed. This leads to lumps or graininess of the gel which can be deleterious to the sensory properties of products in which the master gel is used as a thickener, e.g., a cosmetic cream or lotion. This incomplete dispersion can also influence the weight efficiency of the master gel in thickening the end use formulation. Thus, it is desirable that the carrier provides an adequate processing window, i.e., does not thicken too quickly. The process window can be determined under controlled conditions as is discussed in the EVALUATION METHODOLOGY section.

A final important property of the carrier is that it should be able to efficiently thicken the variety of oils found in different end-use applications without introducing unwanted side effects like graininess, opacity, physical separation, or other sensory properties, e.g., excessive greasiness. For cosmetic applications the key oils are ester oils like triglycerides and fatty esters, hydrocarbons such as mineral oil or branched alkanes, and silicones especially volatile silicones.

It has been found through extensive experimentation that carriers composed of a substantially dissolved mixture of at least one silicone fluid and at least one organic oil induce a high degree of swelling and delamination of organoclays, provide an adequate process window, and are compatible with a range of cosmetic oils and vehicles.

The term “substantially dissolved mixture” is used herein to designate mixtures in which at least about 90%, preferably greater than 95% and most preferably greater than 98% of each of the component fluids used, i.e., the silicone fluid(s) and organic oil(s) is present in a single phase solution at the concentrations employed in the mixture. Ideally, the mixture should be a clear single phase solution. However, cosmetic grade oils, e.g., triglycerides are often mixtures of different molecular weights and chemical species (saturated Vs unsaturated fatty acids). Hence, constituent oils that form hazy solutions in which the majority of species are dissolved but a minority portion of species (e.g., less than 5%) is in dispersed form may be acceptable provided that these undissolved species do not detract from the properties of the finished products in which the master gel is employed as a thickener.

Silicone Fluids

Preferred silicone fluids are cyclic or linear polydimethylsiloxane fluids having a relatively low molecular weight as characterized by their viscosity. The names of polydimethylsiloxane fluid, silicone fluid, silicone oil, and silicone are used interchangeably in the present invention. The preferred silicones have a viscosity less than about 500 cP, preferably less than 50 cP, more preferably less than 30 cP and most preferably less than 10 cP.

Preferred low viscosity silicone fluids include linear and cyclic volatile methyl siloxanes, linear and cyclic volatile and non-volatile siloxanes. These linear and cyclic siloxanes may also be branched.

Most preferred silicone fluids are volatile low molecular weight linear and cyclic polydimethylsiloxanes (VMS) that have viscosity below about 10 cPs, including, for examples, octamethyltrisiloxane, cyclotetrasiloxane, cyclopentasiloxane, and commercial silicone fluids such as DM-fluid-1.5cs, -2cs, -5cs, -A-6cs, and -10cs of ShinEtsu; and DC 245 and DC 2-1184 from Dow Corning.

The silicone fluid component or components typically makes up between about 10% and about 95% of the carrier by weight of carrier, preferably 50% to 90%, and most preferably 60% to 90%.

Organic Oil

The organic oils preferred as components of the carriers of the instant invention are those which are suitable for cosmetic and personal care application, e.g., are mild and safe for contact with human skin. Especially suitable organic oils are ester oils, hydrocarbon oil, and fluorinated hydrocarbon oils in which some or all of the hydrogen atoms are replaced by fluorine.

Ester oils include natural oils (vegetable or animal derived) especially mon-, di- and triglycerides and their mixtures. Non-limiting examples of natural oils include jojoba oil, sunflower oil, soybean oil, canola oil, cottonseed oil, meadow foam seed oil babasu oil, palm oil, avocado oil, fish oil, and the like and various mixtures thereof.

Non-limiting examples of synthetic ester oils include the various fatty acid esters like isopropyl palmitate, isopropyl myristate, ethylhexyl palmitate and isononyl isononoate; C₁₂₋₁₅ alkyl benzoate, propylene glycol dibenzoate, lauryl lactate, triethylhexyl citrate, caprylic/capric triglyceride, neopentyl glycol dicaprylate/dicaprate, octyldodecyl neopentanoate, octyldodecyl isostearate, trioctyidodecyl citrate, polypropylene glycol esters such as PPG-2 myristyl ether proprionate, PPG-3 benzyl myristate and the like and mixtures thereof.

Non-limitiing examples of hydrocarbon oils include mineral oil, paraffin oil, linear or branched alkanes, linear or branched alkenes, isoparaffins, isohexadecane, isododecane, polybutenes, polyisobutene, polyalphaolefin, and various other linear, branched and cyclic paraffinic and napthinic hydrocarbons useful in cosmetic applications.

Fluorinated hydrocarbon oils can also be incorporated in the carrier. Examples of suitable fluorinated oils include FOMBLIN from Montefluos, SILUBE GME-F from Siltech.

The organic oils of this invention also include organo-modified polydimethylsiloxane fluids where part of the methyl groups on the polydimethylsiloxane have been replaced with phenyl, alkyl of higher than methyl, or other organic groups. The terms of organo-modified polydimethylsiloxane, organosilicone(s), and oganosiloxane(s) of this invention are used interchangeably. The organosiloxanes are characterized by their excellent solubility or compatibility with both silicone oils and purely carbon hydrocarbon based organic oils. Examples of the preferred organo-modified silicones include phenyl trimethicone, hexyl methicone, caprylyl methicone, stearyl methicone, lauryl methicone, caprylyl trimethicone, stearoxyltrimethylsilane, trimethylsilyl trimethylsiloxyl lactate, etc.

The preferred oils are those with viscosity of less than 50 cPs, and the most preferred ones are those with viscosity of less than 30 cPs.

The proportions of organic oils used in the carrier mixture ranges from about 5% to 90%, preferably from 10% to 60%, and most preferably from 10% to 50%. It has been found that when the proportions of organic oils is less than 5% in the oil, the master gel is relatively less effective in providing thickening power. In contrast when the organic oil content is higher than about 50%, it may begin to affect the end use application for which the inventive master gel is intended, e.g., alter the sensory properties or compatibility of the master gel with the cosmetic composition used.

Polar Activators

As discussed above, the organoclays are normally in the form of agglomerated platelet stacks. When sufficient mechanical and/or chemical energy is applied to the stacks, the stacks can be delaminated or exfoliated. Some organoclays are referred to as self-activating; that is, no or a smaller amount of polar activators are required to achieve a full exfoliation and dispersion in the solvent. Other clays, which are not self-activating, would require in the master gel an appropriate amount of polar activators to achieve adequate exfoliation. These polar activators act by migrating into two platelets of clay, causing them to swell apart. This reduces the attractive forces between the platelets, so that mechanical energy (shear) can break them apart.

Examples of polar activators include acetone, methanol, ethanol, formaldehyde, and propylene carbonate. However, the most suitable activators for this invention are safe to skin and not highly flammable and include; propylene carbonate, butylene carbonate, propylene glycol, ethanol, ethyl lactate, propyl lactate, butyl lactate, trimethyl citrate, glycerol triacetate, mixtures thereof and aqueous solutions thereof.

Optional Ingredients

Optional ingredients include preservatives (propyl paraben, phenoxyethanol, etc.), antioxidants (BHT, BHA, etc.), perfumes, colorants, etc. The optional ingredients also include cosmetically acceptable semi-solid or solid material as long as their presence in the carrier is within the criteria of “substantially dissolved mixture” as defined above. Examples include C₂₀₋₂₄ alkyl dimethicone, stearyl stearate, etc.

Process of Preparing Master Gels

The preparation of organoclay dispersions is well known in the art and generally includes an intensive mixing step. Suitable processing equipments for intensive mixing include various high-speed, shaped blade intensive mixers such the COWLES DISPERSER or ULTRA-TURRAX mixer, single-stage and multi-stage colloid mills (rotor/stator and/or gear tooth type mills), valve homogenizers, ultrasonic dispersers and the like, used alone or in combination.

There are a number of applicable processes. For example, a one-step process wherein the organoclay is mixed with carrier oils followed by addition of the required amount of polar activators with continued mixing using any of the above described intensive mixers until the desired master gel paste is achieved.

Alternatively a two-step process can be employed. Here, one may partially exfoliate the organoclays in the carrier oils by processing a mixture of organoclays, oils, and a partial amount of polar activators using any of the above intensive mixing techniques. This is followed by the addition of the remaining amount of polar activator while mixing/processing the mixture to a paste like consistency to obtain the desired master gel.

The master gels of this invention are pastes with a range of “hardness values” or viscosity depending upon the level of organoclay, composition of the carriers, activators, and processing conditions, etc. Preferred master gels of the invention have a viscosity of at least 2.5 million cP, as measured by the method described below in the EVALUATION METHODOLOGY section.

Evaluation Methodology

Viscosity Measurements

The viscosity of the master gels was measured using a Brookfield viscometer fitted with a Helipath spindle TF and at 1 rpm. Viscosities at three sample locations were averaged to obtain the reported viscosity for the sample. At each sample location, a data point was recorded every 15 second as the spindle TF traveling down the sample, and a total of seven data points were averaged to obtain the viscosity at that location in the gel.

The viscosity of the thickened oil was also measured using a Brookfield viscometer but with regular RV spindles #3, 4, or 5. Since the thickened oil always exhibited a strong shear-thinning rheology, its viscosity was measured over a range of shear rate as expressed by rpm.

Determination of Process Window

Before the addition of polar activators, the mixture of the organoclays and the carrier was normally very fluid; visually, it flowed very easily and rapidly around the beaker and the splashing sound was very audible as it was being mixed by the propeller. After the addition of polar activators, the timer started; and the viscosity would increase, the flow would slow down, and the splashing sound would decrease. The time when the flow began to slow down visibly and the splashing sound also decreased in parallel was taken as T₁. The time when the flow and the splashing sound stopped was taken as T₂. The desired process window is to have a reasonably long T₁ (e.g., greater than 30 sec in intensive mixing) to allow thorough mixing of the activators into the fluid and a reasonably short time of T₁ minus T₂ (e.g., less than 50 seconds).

Determination of Thickening Efficiency

The typical levels of use of the master gel of this invention depend on the master gel formulation and end-use application. Emulsion type products may require about 3 to about 15% addition while single oil phase product may require from about 10% to about 35% by weight of finished cosmetic composition.

For the purpose of comparing the thickening effectiveness of the master gel of this invention with that of commercial master gel products, we first prepared a 20% solutions of the master gels in suitable cosmetic oils by mixing them together with an overhead stirrer for up to 60 minutes until the dispersion became homogeneous. After measurement of viscosity (Brookfield viscometer (Model: DVII plus pro) using RV spindles following the procedure described above), the mixture is diluted to 15% and 10% with the test oil and their viscosities measured. These viscosity measurements of various dilutions provide an objective basis for comparing the efficiency of various master gel compositions.

Applications

The master gels of the invention can thicken the oil phase of many finished products such as lip sticks, eye make-up, pressed powder, nail polish, hair preparation, deodorants, foundations, hair conditioners, hair styling aids, skin lotions and creams. Although most useful for the anhydrous oil continuous composition, the master gels of the invention can also be used to thicken the oil phase in water-in-oil emulsions, water-in-silicone emulsions, oil/silicone-in-water emulsions, and water-in-oil-in-water multiple emulsions. The water phase of these emulsions may include polyol such as glycerol, butylene glycol, propylene glycol, etc.

### EXAMPLES

The following examples are shown as illustrations of the invention and are not intended in any way to limit its scope.

### Example 1

This example illustrates the preparation of the gelling concentrates or master gels of the present invention and their properties compared with prior art compositions.

The compositions of Examples Ex1A-Ex1D and comparative examples C1A and C1B are shown in Table 1. These compositions were prepared as follows:

The batch sizes were from 250 g to 500 g. The carrier phase was first prepared in a stainless steel beaker of 500 ml to 1000 ml and mixed until uniform. The organoclay was then slowly added to the beaker and mixed with an overhead mixer fitted with a propeller blade for 30 minutes to form a premix. The premix was then homogenized by means of a COWLES DISPERSER for a few minutes. The activator (propylene carbonated in this case) was then added all at once and a timer started (T=0). The time at which the viscosity began to increase (onset of gelation) was estimated visually, designated T₁, and recorded. The viscosity continued to increase as mixing was continued and the time at which gelation appeared complete was recorded and designated T₂. From this point forward, mixing became very difficult, requiring a strong mixing force. Mixing was stopped shortly after time T₂ was reached. The gel viscosity was then measured employing of Brookfield viscometer fitted with a TF spindle at 1 rpm as described above. The gelation time range T₁-T₂ and the Brookfield viscosity were recorded. The results are given in Table 1

Table 1 indicates that the gelling times and ultimate gel viscosity of organoclays depends on the composition of the carrier. For purely organic carries, e.g., comparative C1B, the reaction is very rapid and in a large reactor, gelation can be too fast to achieve a homogenous mixing of the organoclays and the activator. In this case the resulting master gel could become inhomogeneous. In contrast, in a purely silicone carrier, comparative C1A, the gel viscosity is relatively low leading to both inferior thickening performance and even phase separation of the organoclay over time.

It is noted in Table 1 that by using a carrier which is a mixture of silicone fluid (a volatile silicone in this case) and organic oil (an ester oil in this case) according to the instant invention, Ex1A to Ex1D, high viscosity gels having adequate working times are achieved. The resulting master gels employing mixed carriers (Ex1A to Ex1D) were observed to be more homogenous and stable and had a higher viscosity and better sensory properties (e.g., less greasy feel) than the comparative gels C1A and C1B which employed either the silicone fluid (volatile silicone) alone or the ester oil (octyl palmitate) alone.

TABLE 1
Compositions and properties of exemplary and comparative examples.

| | Examples | | | | | | |
|---|---|---|---|---|---|---|---|
| | C1A | | | | | | C1B |
| | (Comparative) | Ex1A | Ex1B | Ex1C | Ex1D | Ex1E | (Comparative) |
| | Wt % | Wt % | Wt % | Wt % | Wt % | Wt % | Wt % |
| ORGANOCLAY | | | | | | | |
| TIXOGEL VPV | 15.10 | 15.10 | 15.10 | 15.10 | 15.10 | 15.10 | 15.10 |
| powder (Sud | | | | | | | |
| Chemie) | | | | | | | |
| CARRIER PHASE | | | | | | | |
| Silicone liquid | 82.33 | 74 | 65.86 | 41.17 | 16.47 | 8.23 | |
| (DC245 volatile | | | | | | | |
| silicone Dow | | | | | | | |
| Corning) | | | | | | | |
| Organic oil - ester oil | | 8.20 | 16.47 | 41.17 | 65.86 | 74.10 | 82.33 |
| (octyl palmitate) | | | | | | | |
| ACTIVATOR | | | | | | | |
| propylene carbonate | 2.57 | 2.57 | 2.57 | 2.57 | 2.57 | 2.57 | 2.57 |
| % SILICONE IN | 100 | 90 | 80 | 50 | 20 | 10 | 0 |
| CARRIER PHASE | | | | | | | |
| Silicone/Organic ratio | 1/0 | 9/1 | 8/2 | 5/5 | 2/8 | 1/9 | 0/1 |
| Gelling time range | 10˜60 min | 30˜50 sec | 20˜30 sec | 20˜25 sec | 12˜25 sec | 10˜25 sec | 10˜25 sec |
| (T1˜T2) | | | | | | | |
| Viscosity, million cP | 0.30 | 1.88 | 2.88 | 3.84 | 3.34 | 3.32 | 2.90 |
| Visual uniformity | phase | Uniform | Uniform | Uniform | Uniform | Uniform | Uniform but |
| | separation | | | | | | darker |
| | over time | | | | | | |

### Example 2

This example illustrates the ability of gel concentrates to thicken both silicone and organic oils.

The master gel compositions described in example 1 were mixed with either a volatile silicone oil (DC 245) or octyl palmitate to form a dispersion containing 20% of master gel. Mixing was accomplished with a GREERCO homomixer until a homogeneous soft paste was obtained. The paste was then allowed to sit at room temperature at least overnight. The viscosity of these dispersions was then measured with a Brookfield viscometer at several shear rates (rpm) settings. The results are collected in Table 2 and Table 3.

It is seen in Table 2, that the exemplary master gels Ex1A-Ex1E incorporating a mixed carrier, thicken the volatile silicones much stronger than a gel utilizing a purely silicone based carrier. Further, the exemplary compositions Ex1C-Ex1E were also superior in thickening the volatile silicone at low shear rates (a measure of suspending ability) than gels utilizing organic oil on its own as the carrier (comparative C1B).

TABLE 2
Effect of composition of carrier oil on performance of master gel in
thickening volatile silicones (composition: 20%
master gel + 80% DC 245)

| | % | | | | | |
|---|---|---|---|---|---|---|
| | Silicone in | Brookfield Viscosity (cP) | | | | |
| Master Gel | Carrier oil | 0.5 rpm | 1 rpm | 5 rpm | 10 rpm | 50 rpm |
| C1A | 100 | Phase separation. Viscosity | | | | |
| (Comparative) | | was too low to measure | | | | |
| Ex1A | 90 | 8,500 | 5,200 | 1,400 | 800 | 212 |
| Ex1B | 80 | 14,500 | 9,500 | 2,200 | 1,250 | 315 |
| Ex1C | 50 | 24,000 | 13,500 | 3,300 | 1,750 | 440 |
| Ex1D | 20 | 26,500 | 14,500 | 6,500 | 1,870 | 476 |
| Ex1E | 10 | 27,500 | 15,000 | 3,600 | 1,960 | 486 |
| C1B | 0 | 23,000 | 13,500 | 3,400 | 1,800 | 456 |
| (Comparative) | | | | | | |

It is seen from Table 3 that the exemplary master gels Ex1C-Ex1E were also significantly more effective in thickening the organic ester oil than either of the single oil carrier gels. It has been found that master gels in which the carrier oil contains from about 10 to about 70% silicone fluid are especially effective in structuring a range of oil based compositions.

TABLE 3
Effect of composition of carrier oil on performance of
master gel in thickening organic oil (composition: 20%
master gel + 80% Octyl palmitate)

| | % | | | | | |
|---|---|---|---|---|---|---|
| | Silicone in | Brookfield Viscosity (cP) | | | | |
| Master Gel | Carrier oil | 0.5 rpm | 1 rpm | 5 rpm | 10 rpm | 50 rpm |
| C1A | 100 | Phase separation. Viscosity | | | | |
| (Comparative) | | was too low to measure | | | | |
| Ex1A | 90 | 15,300 | 8,700 | 2,150 | 1,150 | 316 |
| Ex1B | 80 | 30,000 | 16,500 | 3,750 | 1,960 | 514 |
| Ex1C | 50 | 57,000 | 29,000 | 6,600 | 3,440 | 930 |
| Ex1D | 20 | 56,500 | 29,000 | 6,500 | 3,460 | 908 |
| Ex1E | 10 | 55,000 | 28,500 | 6,500 | 3,460 | 900 |
| C1B | 0 | 47,000 | 24,000 | 5,400 | 2,860 | 694 |
| (Comparative) | | | | | | |

### Example 3

This example shows that dimethicone fluids of viscosity up to 500 cP can be used as components of the carrier of the present invention. In general, volatile silicones and dimethicone fluids of viscosity less than 50 cP are very compatible with the organic oils in all proportion, resulting in a clear mixture, which is the preferred carrier of the present invention. Dimethicone fluids of viscosity from 60 cp to 500 cP can also form a clear mixture with organic oils or solvents within certain compositions. The exact composition that provide a compatible clear carrier depends upon the nature of the organic oils or solvents utilized. However, as long as they form a substantially dissolved mixture, i.e. are predominantly compatible on a molecular level, they can be utilized in the present invention. This is illustrated in the following examples.

A series of mixtures of various silicone fluids of different structure (cyclic and linear) and viscosities (4cP-1000 cP) were prepared with octyl palmitate as the organic oil at different mixing ratios. The mixtures and their properties are summarized in Table 4.

TABLE 4
Compatibility of silicones of various molecular weights with octyl palmitate (OC).

| | | Silicone/OP Ratio | | | | |
|---|---|---|---|---|---|---|
| | | 4/1 | 2/1 | 1/1 | 2/3 | 1/2 |
| | | % Silicone | | | | |
| | Silicone | 80 | 66 | 50 | 40 | 33 |
| Silicones fluid | viscosity (cP) | Compatibility | | | | |
| DC245 | 4 | clear | clear | clear | clear | clear |
| DC200 | 5 | clear | clear | clear | clear | clear |
| DC200 | 50 | clear | clear | clear | clear | cloudy |
| DC200 | 100 | clear | cloudy | separated | separated | separated |
| DC200 | 200 | clear | cloudy | separated | separated | separated |
| DC200 | 350 | clear | cloudy | separation | | separation |
| DC200 | 500 | clear | cloudy | separation | | separation |
| DC200 | 1000 | cloudy | cloudy | separation | | separation |

DC 245 from Dow Corning is a mixture of volatile cyclicsiloxanes
DC 200 from Dow Corning is a series of dimethicone oils having different molecular weights yielding different viscosities.
DC245 and DC 200 with <55 cP are classified as volatile silicones.

A master gel composition, designated as Ex3, consisting of 21.1 % TIXOGEL VP-V organoclay powder (Sud Chemie), 37.59% of a dimethicone oil, DC 200, of 50 cP viscosity (Dow Corning), 37.5% octyl palmitate, and 3.61 % propylene carbonate 15 was prepared by the same method as described in Example 1.

The Ex3 master gel was combined at a 20 wt % level with the volatile silicone DC 245. As shown in Table 5 below, the thickening performance of this master gel employing a low viscosity dimethicone was highly satisfactory.

TABLE 5
Performance of Ex3 master gel containing dimethicone
in thickening a volatile silicone (DC 245).

| | shear rate, rpm | | | | |
|---|---|---|---|---|---|
| | 0.5 | 1 | 5 | 10 | 50 |
| Brookfield viscosity, cP | 30,000 | 16,000 | 4,000 | 2,300 | 580 |

### Example 4

This example illustrates master gels of the present invention at high loading of organoclays and further demonstrates the benefits of employing the mixed carriers identified herein for these compositions.

A series of master gel compositions identified in Table 6 were prepared by the methods used in Example 1. The compositions differed in the level of organoclay used and in the composition of the carrier.

As the organoclay content of the gels increases the working times of T₁ and T₂ for homogeneously mixing the activators into the slurry decrease rapidly. This can make the preparation of master gels at an organoclay content of more than 10% quite difficult, especially when the gel's viscosity exceeded 2 million cP.

However, using the mixed carriers of the present invention the working times are increased sufficiently to prepare uniform gels (Ex4A and Ex4B). The optimum composition in terms of the organoclay load and the ratio of dimethicone fluid to the organic oils depends on the nature and selection of the dimethicone fluids and the organic oils, on the nature of the activator, and on the processing equipments. Recognizing the principles set forth in the instant invention, however, now allows optimal compositions to be prepared with minimum experimentation by systematically determining working time and gel viscosity as a function of composition.

TABLE 6
Influence of carrier on mater gels prepared with high levels of organoclay

| | Exemplary and | | | | | | | |
|---|---|---|---|---|---|---|---|---|
| | comparative | | | | | | | |
| | examples | | | | | | | |
| | | | | | | | Exemplary | |
| | Comparative Compositions | | | | | | Compositions | |
| | C4A | C4B | C4C | C4D | C4E | C4F | Ex4A | Ex4B |
| | Wt % | Wt % | Wt % | Wt % | Wt % | Wt % | Wt % | Wt % |
| ORGANOCLAY | | | | | | | | |
| TIXOGEL VPV | 10 | 15 | 20 | 30 | 15 | 20 | 20 | 30 |
| powder | | | | | | | | |
| CARRIER | | | | | | | | |
| DC 245 | | | | | 82.75 | 77 | 50.05 | 55.68 |
| Octyl palmitate | 88.50 | 82.75 | 77 | 65.50 | | | 26.95 | 9.83 |
| ACTIVATOR | | | | | | | | |
| propylene | 1.50 | 2.25 | 3 | 4.50 | 2.25 | 3 | 3 | 4.50 |
| carbonate | | | | | | | | |
| % SILICONE IN | 0 | 0 | 0 | 0 | 100 | 100 | 65 | 85 |
| CARRIER | | | | | | | | |
| T₁ | 50 sec | 20 sec | 15 sec | 8 sec | 5 min | 1.5 min | 20 sec | 10 sec |
| T₂ | 12 min | 1 min | 25 sec | 10 sec | 25 min | 5 min | 25 sec | 14 sec |
| Gel viscosity, | 0.45 | 2.19 | 4.58 | >10 | 0.08 | 1.23 | 5.72 | >10 |
| million cP | | | | | | | | |

### Example 5

This example illustrates that the master gels of the invention employing mixed carries are capable of thickening a variety of oils that are useful for various applications in addition to silicone fluids. The example also illustrates that the thickening performance of the master gels of the invention are much more efficient than the commercially available master gel concentrates.

Mixtures of the exemplary master gels of Example 4, Ex4A and Ex4B, and various commercially available gel concentrates were prepared with four different classes of oils. The oils were a volatile silicone (DC 245), C₁₂₋₁₅ alkyl benzoate (synthetic ester oil), jojoba oil (natural plant oil), and mineral oil (synthetic hydrocarbon oil). The commercially available master gels included TIXOGEL VSP-1438, TIXOGEL FTN, TIXOGEL JOG-1583, and BENTONE GEL MIOV. The level of master gel used was 20%, 15%, or 10% based on the total weight of the final mixture (master gel+oil). The results are collected in Table 7.

TABLE 7
Comparative performance with the commercial master gel products

| | | | Brookfield Viscosity | | | | |
|---|---|---|---|---|---|---|---|
| | Oil to be | Wt % | 0.5 | | | | |
| Master Gel | thickened | master gel | rpm | 1 rpm | 10 rpm | 50 rpm | 100 rpm |
| TIXOGEL | Volatile silicone | 20 | 10,500 | 6,000 | 770 | 204 | 120 |
| VSP-1438 | DC245 | | | | | | |
| Ex4A | Volatile silicone | 15 | 21,200 | 11,900 | 1,650 | 416 | 241 |
| | DC245 | | | | | | |
| Ex4A | Volatile silicone | 12 | 11,000 | 6,150 | 880 | 239 | 142 |
| | DC245 | | | | | | |
| Ex4B | Volatile silicone | 15 | 18,500 | 10,840 | 1,480 | 380 | 220 |
| | DC245 | | | | | | |
| TIXOGEL | C₁₂₋₁₅ alkyl | 20 | 13,000 | 6,700 | 976 | 293 | 183 |
| FTN | benzoate | | | | | | |
| Ex4A | C₁₂₋₁₅ alkyl | 10 | 50,000 | 40,000 | 5,200 | 1,260 | 720 |
| | benzoate | | | | | | |
| TIXOGEL | Jojoba oil | 20 | 43,000 | 22,000 | 3,100 | 860 | 534 |
| JOG-1583 | | | | | | | |
| Ex4A | Jojoba oil | 15 | 28,000 | 15,000 | 2,370 | 992 | 554 |
| Ex4B | Jojoba oil | 15 | 56,000 | 30,000 | 4,050 | 1,116 | 683 |
| BENTONE | mineral oil | 20 | 11,500 | 6,000 | 930 | 290 | 188 |
| GEL MIOV | | | | | | | |
| Ex4A | mineral oil | 10 | 24,000 | 12,500 | 1,750 | 518 | 320 |
| Ex4B | mineral oil | 10 | 92,000 | 51,000 | 7,100 | 1,888 | 940 |

Each of the four commercial master gels is designed separately by the manufacturers for thickening primarily its carrier oil. Surprisingly, the master gels of the present invention were found to be able to thicken a wide range of organic oil types as well as the volatile silicone fluids. For example, Ex4A of the present invention alone is universally effective in thickening all four classes of oils. In contrast, four different commercial master gels are required to cover the four classes of oils. This example further illustrates the unexpected universal nature of the master gels of the present invention for thickening silicone and organic oils.

More surprisingly, the inventive gel (Ex4A or EX4B) alone provided much higher suspending/thickening power than any of the commercial gels for the oil(s) that each of these commercial gels was specifically designed for. For examples, 12% of Ex4A of the present invention was as efficient as 20% of the commercial TIXOGEL VSP for thickening the volatile silicones; it improves the formulation space by 8%. 10% of Ex4A was more efficient for thickening C₁₂₋₁₅ alkyl benzoate than 20% of the commercial TIXOGEL FTN, providing to the formulators an extra 10% of the formulation space. Ex4A or Ex4B of the present invention at 10% were able to thicken mineral oil better than 20% of the commercial BENTONE Gel MIOV, providing an extra 10% of the formulation space. The saved formulation space from the superior efficiency of the inventive gels can now be used for the inclusion of other critical actives or components.

Thus, the inventive gels provide very substantial improvements in both the efficiency and effectiveness in all kinds of oils compared with commercially available thickening master gels.

### Example 6

The compositions shown in Table 8 illustrate the wide range of master gel compositions that can be prepared within the scope of the present invention.

TABLE 8
Examples of master gels of the present invention

| | Ex 6A | Ex 6B | Ex 6C | Ex 6D |
|---|---|---|---|---|
| EXEMPLARY MASTER GEL | Wt % | | | |
| ORGANOCLAY | | | | |
| TIXOGEL VPV powder | 30.35 | 30.35 | 33.00 | 15 |
| CARRIER OIL | | | | |
| DC 245 | 38.70 | 38.70 | 43 | 41.25 |
| Jojoba oil | 25.80 | | 6.13 | |
| Mineral oil | | 25.80 | 6.13 | |
| Caprylic/capric triglyceride | | | 6.13 | |
| isododecane | | | | 41.25 |
| ACTIVATOR | | | | |
| propylene carbonate | 5.15 | 5.15 | 5.60 | 2.50 |
| % Silicone oil in carrier | 60 | 60 | 70 | 50 |
| Gel viscosity in million cP | >10 | >10 | >10 | 2.29 |

### Example 7

This example illustrates that the master gel of a wide range of organoclays and different kinds of activators can be prepared within the scope of the present invention.

TABLE 9
Exemplary master gels

| EXEMPLARY MASTER GEL | | | |
|---|---|---|---|
| INGREDIENTS | Ex 7A | Ex 7B | Ex 7C |
| ORGANOCLAY | | | |
| TIXOGEL LGM | 15 | | |
| Nanomer I.44P | | | 20 |
| Cloisite 15A | | 18 | |
| CARRIER OIL | | | |
| DC 245 | 66.20 | 56.14 | 46.2 |
| Ethylhexyl palmitate | | | 30.8 |
| Butyl acetate | 16.55 | 24.06 | |
| ACTIVATOR | | | |
| Propylene carbonate | 2.25% | | |
| Methyl lactate | | 2.55 | 3 |
| % Silicone oil in carrier | 80 | 70 | 60 |
| Gel viscosity in million cP | 7.19 | 2.13 | 6.11 |

### Example 8

This example illustrates the use of organo siloxanes (phenyl trimethicone, stearoxytrimethylsilane, and C₂₀₋₂₄ alkyl dimethicone) as a class of organic oil for the mixed carriers of the present invention. The C₂₀₋₂₄ alkyl dimethicone is a waxy solid material at room temperature and is not totally soluble with other carriers; it serves to illustrate that as long as the mixture of the carrier oils is a “substantially dissolved mixture”, the carrier mixture would work within the scope of the present invention.

The batch size of this gel was 260 g. The carrier was prepared by mixing DC245 (48.94%), phenyl trimethicone (7.89%), ethylhexyl palmitate (3.55%), stearoxytrimethylsilane (15%) and C₂₀₋₂₄ alkyl dimethicone (3.55%) in a beaker, followed by warming it to 70° C. to dissolve the waxy alkyl dimethicone into a clear solution. The resulting carrier at the room temperature was a translucent solution of “substantially dissolved mixture”. TIXOGEL VPV (18%) and the above carrier were blended with a propeller for 30 minutes, followed by adding the propylene carbonate (3%) to form the gel. The viscosity of the resulting master gel 4.3 million cP. This gel showed an extremely silky and lubricious skin sensory when it was rubbed over the skin.

### Example 9

This example illustrates the application of the gels of the present invention in the area of water in silicone emulsion or water in oil emulsion formulations. This water-in-oil liquid make-up product whose composition is recorded in Table 9A employed the master gel composition Ex6A (Table 10A) of the present invention and was produced by the manufacturing procedures described below. It was a rich and lubricious cream, which was spreaded over and blended into the skin readily. Its viscosity decreased significantly from 0.5 rpm to 100 rpm (Table 10B), demonstrating the benefits of the master gels of the present invention—a unique combination of superior suspending power and silky skin sensory.

TABLE 10A
Water-in-oil emulsion liquid make-up formulation

| Ingredients | % by weight | Phase |
|---|---|---|
| Ex 6A Mater gel (see Table 8) | 3.5% | A |
| DC 245 | 12% | A |
| Cetyldimethicone copolyol | 3.5% | A |
| Octyldodecyl myristate | 3% | A |
| Versagel MP 750¹ | 3% | A |
| Isodocecane | 11% | A |
| Isononyl isononoate | 5% | A |
| Cetyl alcohol | 3% | A |
| Black iron oxide, dimethicone coated | 0.03% | B |
| Red iron oxide, dimethicone coated | 0.22% | B |
| Yellow iron oxide, dimethicone coated | 0.6% | B |
| Titanium dioxide, rutile | 6.15% | B |
| glycerine | 4.9% | C |
| Magnesium sulfate | 0.25% | C |
| Natrosol CS Plus² | 0.49% | C |
| Liquid Germal plus³ | 0.03% | C |
| water | To 100% | C |

¹from Penreco.
²modified hydroxyl cellulose from Hercules.
³Preservative, from ISP

TABLE 10B
Viscosity profile of the W/O liquid make-up formulation

| | Share rate | | | | | |
|---|---|---|---|---|---|---|
| | 0.5 rpm | 1 rpm | 5 rpm | 10 rpm | 50 rpm | 100 rpm |
| Viscosity, cP | 127,000 | 97,000 | 20,000 | 10,000 | 3,000 | 2,000 |

Manufacturing procedures:

1) The phase B pigments were blended with a small kitchen blender to a homogeneous powder.

2) The phase C ingredients were added to a beaker using a propeller to a homogeneous aqueous mixture.

3) The phase A were added in the order given in Table 10A into a stainless beaker, and homogenized with a rotor/stator colloid mill to a homogeneous paste.

4) Phase B mixture was added to the mixture formed in step 3) and homogenization was continued until the mixture became a homogeneous color paste.

5) The temperature was raised to 65° C., and the phase A mixture was added with continued mixing.

6) The phase C aqueous solution was added in portions into the beaker while maintaining the mixing and the temperature above 65° C.

7) After the completion of the phase C addition, the heater was turned off while continuing the mixing for about an additional 20 minutes.

8). The mixture was poured into a container and cooled to room temperature.

### Example 10

This example illustrates the application of the gels of the present invention in the area of oil-in-water emulsion products using the master gel prepared in Example 8. Phase A was prepared by mixing jojoba oil in portions into the master gel using an overhead mixer. Phase B was prepared by mixing all components with an overhead mixer for 10 minutes. Phase A was slowly added into phase B at room temperature while mixing vigorously with the overhead mixer. The final product was a very light and soft cream. It rubbed into skin quickly and left a soft feel on skin.

TABLE 11
Oil-in-water daily skin moisturization lotion

| Ingredients | Wt % | Wt (gm) | Phase |
|---|---|---|---|
| Master gel of Example 8 | 3% | 9 | A |
| Jojoba oil | 10% | 30 | A |
| glycerine | 4% | 12 | B |
| Liquid Germal Plus | 0.5% | 1.5 | B |
| Viscolam AT 100P | 1.6% | 4.8 | B |
| water | 80.9% | 242.7 | B |

While this invention has been described with respect to particular embodiments thereof, it is apparent that numerous other forms and modifications of the invention will be obvious to those skilled in the art. The appended claims and this invention generally should be construed to cover all such obvious forms and modifications which are within the true spirit and scope of the present invention.

What is claimed is:

1. A master gel composition comprising:
i) greater than about 10% to about 40% of an organoclay,
ii) from about 60% to less than about 90% of a carrier oil comprising a substantially dissolved mixture of at least one silicone fluid and at least one organic oil wherein the total silicone fluids comprise from about 10% to about 90% of the carrier based on the total weight of the carrier,
iii) from 0.5% to about 6% of a polar activator.

2. A master gel composition according to claim 1 wherein the organoclay is Bentonite modified with a quaternary ammonium salts containing methyl, or benzyl radical, and a mixture of dialkyl or trialkyl radicals having from 14 to 30 carbon atoms wherein the organoclay is a non self-activating organoclay, a self-activating organoclay, or a mixture thereof.

3. A master gel composition according to claim 1 wherein the level of organoclay is in the range from about 15% to about 30% and is chosen so that the viscosity of the master gel composition is at least about 2.5 million cP as measured with a Brookfield viscometer at 1 rpm.

4. A master gel composition according to claim 1 wherein the polar activator is selected from the group consisting of propylene carbonate, butylene carbonate, propylene glycol, ethyl lactate, propyl lactate, butyl lactate, trimethyl citrate, glycerol triacetate, isopropanol, ethanol, mixtures thereof and aqueous solutions thereof.

5. A master gel composition according to claim 1 wherein the silicone fluid is selected from the group consisting of volatile and non-volatile branched, non-branched, linear or cyclic silicone fluids and mixtures thereof.

6. A master gel composition according to claim 5 wherein the silicone fluid has a viscosity of about 500 cP or less.

7. A master gel composition according to claim 5 wherein the silicone fluid has a viscosity of about 50 cP or less.

8. A master gel composition according to claim 5 wherein the silicone fluid has a viscosity of about 30 cP or less.

9. A master gel composition according to claim 1 wherein the silicone fluid comprises from about 50% to about 90% of the carrier composition by weight.

10. A master gel composition according to claim 1 wherein the silicone fluid comprises from about 60% to about 90% of the carrier composition by weight.

11. A master gel composition according to claim 1 wherein the organic oil is selected from the group consisting of hydrocarbon oils, ester oils, fluorinated hydrocarbons, naturally-derived plant or animal oils, organosilicones, and mixtures thereof.

12. A master gel composition according to claim 11 wherein the hydrocarbon oil is selected from the group consisting of mineral oil, paraffin oil, isoparaffins, isohexadecane, isododecane, polybutene, polyalphaolefin, polyisobutene, and mixtures thereof.

13. A master gel composition according to claim 11 wherein the ester oil is selected from the group consisting of synthetic or natural mono-, di- or triglycerides, fatty acid esters, C₁₂₋₁₅ alkyl benzoate, propylene glycol dibenzoate, lauryl lactate, triethylhexyl citrate, trioctyldodecyl citrate, caprylic/capric triglyceride, neopentyl glycol dicaprylate/dicaprate, isononyl isononoate, octyldodecyl neopentanoate, PPG-2 myristyl ether proprionate, PPG-3 benzyl myristate, and mixtures thereof.

14. A master gel composition according to claim 11 wherein the naturally derived plant or animal oil is selected from the group consisting of jojoba oil, sunflower oil, soybean oil, canola oil, cottonseed oil, meadow foam seed oil, babasu oil, palm oil, avocado oil, fish oil, and the like and various mixtures thereof.

15. A master gel composition according to claim 11 wherein the organosilicone is selected from the group consisting of phenyl trimethicone, caprylyl trimethicone, C₆ to C₂₀ alkyl methicone, stearoxytrimethylsilane, and mixtures thereof.

16. A method of thickening a cosmetic composition comprising the step of mixing the master gel according to claim 1 with the cosmetic composition to be thickened to form a mixture wherein the master gel comprises from about 1% to about 40% by weight of the mixture.
